(19) United States
(12) Patent Application Publication
(10) Pub. No.: US 2020/0155723 A1
(43) Pub. Date: May 21, 2020

(54) HIGHLY HYDROPHILIC COATINGS FOR BIOMEDICAL APPLICATIONS

(71) Applicant: FUNDAClÓN CIDETEC, DONOSTIA-SAN SEBASTIÁN (ES)

(72) Inventors: Iraida LOINAZ BORDONABE, Donostia-San Sebastian (ES); Laura SÁNCHEZ ABELLA, Donostia-San Sebastian (ES); Hans-Jürgen GRANDE, Donostia-San Sebastian (ES)

(21) Appl. No.: 16/612,305

(22) PCT Filed: May 15, 2018
(86) PCT No.: PCT/EP2018/062594

§ 371 Date: Nov. 8, 2019

(30) Foreign Application Priority Data
May 16, 2017 (EP) 17382277.6

(51) Int. Cl. A61L 27/34

(52) U.S. Cl. CPC A61L 27/34; A61L 2420/06; A61L 2420/02

(57) ABSTRACT

The invention provides a partially or totally coated substrate, the coating comprising a water-absorbing hydrophilic random polymeric network comprising hydrophilic cross- linked polymeric chains which are directly bound to the surface of the substrate, said polymeric chains comprising one or more hydrophilic monomer(s). The invention also provides a process for obtaining the coated substrate of the invention. The coated substrate of the invention shows a long-lasting wear and abrasion protective effect.

This application claims the benefit of European Patent Application EP17382277.6 filed on May 16, 2017.

### TECHNICAL FIELD

The present invention provides coatings for articles such as medical devices, especially artificial implants and other devices fabricated for use inside the body, and methods for their preparation.

### BACKGROUND ART

Artificial joint replacement is an alternative for the substitution of damaged joints that has been employed longer than 50 years now. Even if it is a well-established procedure with low rates of revision, aseptic loosening, dislocation and infection remain principal causes of revision.

Aseptic loosening occurs when wear particles, detached from the prosthesis, activate the immune response. This effect was mainly observed in prosthesis manufactured of ultrahigh molecular weight polyethylene (UHMWPE). This material has been widely used as a bearing surface due to its high strength, high biocompatibility, low friction coefficient and high wear resistance. However, in long-term implantations submicron particles are generated in vivo due to the wear of the prosthesis. When this occurs, macrophages undergo phagocytosis and secretion of bone resorptive cytokines, inducing osteolysis and, in hence, loosening of the implanted prosthesis.

Several strategies have been developed to reduce this effect. The cross-linking of the polyethylene, for example with gamma irradiation, generates a stronger material that reduces the wear. The addition of vitamin E in the UHMWPE prevents from oxidation and consequently from debris. Alternatively, ceramic materials have also demonstrated a good performance notwithstanding that concern is still related to ceramic brittleness. In spite of all these strategies are being widely used at the moment, long term behavior of those materials is difficultly predictable and real in vivo behavior can only be determined after several years of implantation.

Several attempts have been disclosed in the prior art to improve wear behavior while maintaining the same mechanical properties of polyethylene.

One approach was developed by the group of Kazuhiko Ishihara, inspired by the lubrication mechanisms of the human natural joints. These researchers proposed the use of a highly hydrophilic coating produced by grafting a polymerizable monomer having a phosphorylcholine group onto the surface of an artificial joint made of UHMWPE, the surface bearing a photoinduced polymerization initiator on the bearing surface of the substrate. The grafting method proposed generated a highly ordered and dense layer of polymer brushes where the polymer chains assume a stretched conformation in water. This conformation attracts water molecules to the surface inducing a hydrodynamic lubrication mechanism.

This same technology has also been applied to other substrates, including intermediate layers, to increase the bonding strength and the density of the polymer coating to metallic, ceramic or polymeric substrates.

Although these authors have demonstrated good stability in long term studies of the poly(MPC) grafted layers, according to D. Xiong et al work, long term friction induces shear of the polymer brushes. It is the presence of these sheared polymer chains that stays in the solution that act as lubricants.

In spite of the above, it has been found that the highly ordered polymer brushes reported in the prior art show several drawbacks.

On one hand, they are not stable enough under friction so they have to be cross-linked. When cross-linked, the role of water in the lubrication of brush-gel resultant is very relevant. Cross-linked polymer brushes have, however, a limited mobility, and would act as boundary lubricant in a mixed lubrication. The absorbed water will promote the formation of a lubricating fluid film.

It has also been reported that the inclusion of cross-linkings decreases hydrophilicity to the polymer brushes-based coatings: the higher cross-linking degree is, the higher loss of hydrophilicity occurs. And this hydrophilicity loss negatively affects to the provision of a hydrated layer that effectively prevents prosthesis' wear. On the other hand, due to their conformation, cross-linked polymer brushes have a very limited swelling capacity, which is restricted to the length of the polymer chain.

In view of the above, therefore, there is still the need of highly wear resistant hydrophilic coatings which are strongly associated to substrate's surface and which overcome all or part of the drawbacks related to the currently known hydrophilic coatings.

### SUMMARY OF INVENTION

As it has been mentioned above, the prior art taught that a strong association can be achieved if the polymerization initiator is firstly anchored (or adsorbed) to the activated surface and then, from the immobilized initiator, the polymerization starts, providing a highly ordered polymer network (i.e., polymer brushes).

The present inventors have found that when a substrate surface was firstly activated and then in situ radical polymerization and cross-linking were performed, a hydrophilic random 3D-network was achieved, comprising hydrophilic polymeric chains which were directly bound to substrate's surface.

The present inventors have found that the binding does not negatively affect to the hydrophilic nature of the network and to the long-lasting effect of the coating as anti-wear and anti-abrasion barrier.

In connection with the above, it was checked whether the binding of the 3D-network to the surface could negatively affect the hydrophilicity of the polymeric coating. As reported below, the coating, once applied, shows a remarkable high hydrophilicity (with contact angles lower than)50° and swelling capability. Therefore, the direct binding of the polymeric network to the surface does not negatively affect the hydrophilic nature of the network.

The present inventors performed different tribometer tests simulating the behavior, in an articulate prosthesis, of different substrates either coated/uncoated. In particular, these tests were performed comparing the behavior in extreme conditions of a combination of a UHMWPE substrate vs an alumina substrate, coating only the alumina with a coating comprising copolymers of poly(ethylene glycol) methacrylate, Mn 360 (PEGMA-360) and poly(ethylene glycol) dimethacrylate, Mn 550 (PEGDMA-550). As it is concluded in Example 15, simulating an articulate prosthesis in harsh conditions (UHMWPE in edge position over the alumina) comprising a part made of UHMWPE and another of alumina, a reduction of about 70% in wear was achieved by just coating the alumina substrate with the hydrophilic random polymeric network.

On the other hand, performing abrasion tribometer tests simulating, as above, the behavior in an articulate prosthesis, it was also confirmed that just coating one of the substrates resulted in a reduction in abrasion of about 55% (see Example 16 below). That is, it substantially reduced the formation of particulates that can give rise to serious side-effects, due to the hydrophilic random polymeric network comprising hydrophilic polymeric chains directly bound to substrate's surface.

That is, the tribological tests provide indicia that a hydrophilic random polymeric network, comprising cross-linked polymeric chains directly bound to the surface of the substrate, improves the wear and abrasion profile of the substrate. The effect provided by the coating is so remarkable that just coating one of the two substrates can be enough to achieve an efficient protective wear and abrasion effect.

The preliminary tribological data were further supported performing hip simulator tests. It was confirmed, again, that if the surface was activated and then, polymerization in situ occurs, forming a hydrophilic random polymer network which was bound to the surface, a reduction in wear of about 80% was achieved. Even including Al₂O₃ abrasion particles, the “protective” effect provided by the coating was still remarkable (the wear rate was about 55% lower).

The present inventors also found that the 3D-network coating of the invention, even comprising cross-linked polymeric chains, had a strong avidity to water. As shown below, the coating swelling capability was observed by atmospheric pressure SEM where images acquired at 100% of humidity, 750 Pa pressure and 1° C. of temperature were about 7-8 μm thick and were reduced about 10-fold by reducing humidity in the chamber (images acquired at 45% of humidity, 311 Pa pressure and 1° C. of temperature were about 0.8-1 μm thick) (Example 26). In another example, a coating of the invention with an original thickness of about 300 nm in a dry form is showed (Example 25).

As it is further shown below (Example 26 and Example 25), the coating of the invention is able to absorb water, becoming a hydrogel, and to increase up-to 30-fold the original thickness of the coating, this thicker coating having a remarkably hydrophilic nature. The hydrogel shows antifouling properties (Example 24).

The formation of a hydrogel is something important because one of the main applications of the coated substrate of the invention is as implant, being needed a hydrated layer that prevents wear and abrasion. And a hydrogel provides said effective anti-wear and anti-abrasion hydrated layer.

This is something surprising and unpredictable in view of the prior art, wherein it was thought that the cross-linking of polymer brushes caused a loss in hydrophilicity. And, due to the highly ordered disposition of the polymeric chains, no remarkable increase in the thickness could be achieved, being the thickness restricted to the length of the polymer chain. The latter having a direct impact on the life of the article made of the coated substrate.

It was also found that the hydrogel, once formed, was also bound to the surface of substrate, not bounding off the substrate. This is due to the fact that the 3D-network comprises cross-linked polymeric chains directly bound to the surface.

In view of the above, the present invention reports, for the first time, a hydrophilic coating with a thickness in the order of “nm” (due to the random disposition of the polymeric chains), comprising a random polymeric 3D-network which is water-absorbing, becomes a hydrogel, expands and increases its thickness up to 30 times and is stable enough to provide an efficient wear and abrasion protection.

Altogether, the present invention means a great advance in the field of coatings, but especially in the field of medical devices, because it is the first time that it is reported a highly hydrophilic random polymeric coating strongly anchored to the surface of a substrate.

Thus, in a first aspect the present invention provides a partially or totally coated substrate, the coating comprising a water-absorbing hydrophilic random polymeric network comprising hydrophilic cross-linked polymeric chains which are directly bound to the surface of the substrate, said polymeric chains comprising one or more hydrophilic monomer(s).

In a second aspect, the present invention provides process for preparing a partially or totally coated substrate according to the first aspect of the invention, which comprises the steps of: (a) subjecting substrate's surface to an activation surface treatment, and (b) partially or totally coating the activated surface resulting from step (a) by:
b.1. performing an in situ radical polymerization starting from a solution comprising the appropriate monomer(s), using a radical polymerization initiator, and
b.2. cross-linking the polymeric chains.

In a third aspect the present invention provides a partially or totally coated substrate obtained by the process as defined in the first aspect of the invention.

And, finally, in a fourth aspect the present invention provides a partially or totally coated substrate as defined in the first aspect of the invention for use in reconstructive medicine.

### BRIEF DESCRIPTION OF DRAWINGS

FIG. 1 Wear rate of the coated UHMWPE following the protocol described in Example 1 with 10% wt. of the mixture of PEGMA-360 and PEGDMA-550 and a molar ratio of 95:5 and ZTA balls using the protocol described in Example 3 in the hip-simulator compared with the control (wear rate of bare UHMWPE and bare ZTA) according to the experimental described in example 17.

FIG. 2 Wettability and contact angle of the coated PEEK-CFR using the coatings described in Table 11compared with bare PEEK-CFR.

FIG. 3 Wettability and contact angle of the coated UHMWPE with the compositions described in Table 12 compared with bare UHMWPE.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

In a first aspect the present invention provides a partially or totally coated substrate, the coating comprising a water-absorbing hydrophilic random polymeric network comprising hydrophilic cross-linked polymeric chains which are directly bound to the surface of the substrate, said polymeric chains comprising one or more hydrophilic monomer(s). For purposes of the present invention, any ranges given include both the lower and the upper end-points of the range. Ranges given, such as concentrations, temperatures, times, and the like, should be considered approximate, unless specifically stated.

The term “partially coated” means that part of the surface of the substrate is coated with the coating comprising the random polymeric network.

The term “totally coated” means that the whole surface of the substrate is coated with the coating comprising the random polymeric network.

The term “substrate” means the object on which the coating is applied. The object is made of a particular material or combination of materials and has a particular shape. In one embodiment the substrate is made of material(s) suitable to be in contact with human body, such as material(s) used in the manufacture of medical devices. In this regard, Example 21 illustrates that the coating does not show cytotoxic effects. Illustrative non-limitative examples of these materials are plastics, in particular thermoplastics, more particularly thermoplastic polyethylene, such as ultrahigh molecular weight polyethylene (UHMWPE) or thermoset resins such as crosslinked polyethylene (XLPE) or composites such as carbon-fiber-reinforced polyetheretherketone (PEEK-CFR); ceramics, such as zirconia-toughened-alumina (ZTA); and metallic alloys, such as CoCrMo alloy, among others. In one embodiment, the substrate is of a material selected from UHMWPE, ZTA, XLPE, carbon-fiber-reinforced polyetheretherketone (PEEK-CFR), and any combination thereof. In another embodiment, the substrate means an object, as defined above, already comprising one or more coating(s) of a nature different from the one of the present invention, said one or more coating (s) being made of polymers able to be activated to the subsequent in situ radical polymerization step. Illustrative non-limitative examples of such one more coatings are trimethoxysilylpropylmethacrylate, 3-(Tri methoxysilyl)propyl acrylate, N[3-(Trimethoxysilyl)propyl]N′(4-vinylbenzyl)ethylenediamine.

The “substrate” can be understood as a component of a device or composition (in the form of a layer, ball, or cylinder, among others) or can be the whole device. In one embodiment, the substrate is a medical device. In another embodiment, the substrate is an implant, particularly a biomedical implant, particularly an artificial orthopedic implant, more particularly an articulate artificial orthopedic implant.

In case that the device comprises several parts, they can be made of the same material, or alternatively of different materials such as those listed above.

As it is provided in the first aspect of the invention, the coating comprises a “hydrophilic random polymeric network comprising hydrophilic polymeric chains”.

The term “random” when referred to the polymeric network means that the polymeric chains forming the network are distributed on the surface of the substrate without following a particular order pattern.

Since the polymeric network comprises hydrophilic polymeric chains, the coating comprising the random polymeric network will be of the same hydrophilic nature.

The term “hydrophilic polymeric network”, refers to those polymeric networks comprising hydrophilic polymeric chains. A polymeric chain is hydrophilic by the chemical nature of the monomer(s) forming the polymer. In this regards, hydrophilic polymers comprise hydrophilic monomers with polar or charged functional groups. In addition, there are several routine methods to determine the hydrophilicity of a polymer network/coating. One of these methods is based on measuring the contact angle of the polymer. This method comprises the application of a droplet of distilled water on the polymer to be tested and the determination of the contact angle between the droplet and the polymer. If the contact angle is lower than 90°, then the polymeric network/coating is hydrophilic. The less the value of contact angle is, the more the hydrophilicity of the network/coating is. The determination of the contact angle can be performed using, for example, a goniometer or a CCD camera, and following manufacturer's instructions. As it is illustrated below, the present inventors measured the contact angle parameter in order to determine the degree of hydrophilicity of several embodiments of coating. As can be seen, the contact angle was, for all the tested polymeric networks forming part of the present invention, substantially below 90°, being indicative that they were remarkably hydrophilic.

The term “monomer” means, as recognized by IUPAC, a molecule that has one or more polymerizable end-groups that can undergo polymerization thereby contributing constitutional units to the essential structure of a macromolecule (PAC, 1996, 68, 2287 (Glossary of basic terms in polymer science (IUPAC Recommendations 1996)) on page 2289).

In one embodiment, the network comprises a monomer having one polymerizable end-group. In this embodiment, a second monomer is needed to achieve the cross-linking of the polymeric chains (i.e. the hereinafter also referred as “cross-linking agent”), providing a heteropolymeric 3D-network. In one embodiment, this second monomer is hydrophilic and has at least two polymerizable end-groups, giving rise to a covalent cross-linking. In one embodiment, the second monomer has two or more functional groups selected from: an acrylic or methacrylic group (such as an acrylate or methacrylate, or an acrylamide group). In another embodiment, the second monomer (cross-linking agent) is selected from the group consisting of: N,N′-methylenebis(acrylamide) (BAm), poly(ethylene glycol) diacrylate (PEGDA), pentaerythritol triacrylate (PETA), glycerol propoxylate (1P0/0H) triacrylate (GPTA), poly(ethylene glycol) dimethacrylate (PEGDMA), di(ethylene glycol) dimethacrylate (DEGDMA), and bis[2-(methacryloyloxy)ethyl] phosphate (BMAP).

In another embodiment, this second monomer is a hydrophobic monomer, and the cross-linking is due to hydrophobic interactions (such as association, aggregation, crystallization, complexation, and hydrogen bonding).

In another embodiment, the monomer comprises two or more polymerizable end-groups and the resulting network is a homopolymeric 3D-network, wherein the homopolymeric chains and the cross-linking comprise the same hydrophilic monomer. In this embodiment, the monomer has a dual effect: it is used for synthesizing the homopolymeric chain and due to the polymerizable end-groups is able of binding the resulting chains.

A “macromonomer” means, as recognized by IUPAC, a macromolecule that has one or more polymerizable end-groups that can undergo polymerization which enable(s) it to act as a monomer molecule. An example of macromonomer is a polymeric chain bearing, at one end, an acrylate or acrylamide group, for instance. In the present invention, the term “monomer” also embraces the term “macromonomer”.

As mentioned above, at least one of the monomers forming the polymeric chains has to be hydrophilic in nature (to confer that nature to the resulting chain). Quantitatively, the hydrophobic/hydrophilic nature of the monomers may be determined according to the log P of the particular monomers, which is sometimes referred to as the octanol-water partition coefficient.

The partition coefficient, abbreviated P, is defined as a particular ratio of the concentrations of a monomer between the two solvents (a biphase of liquid phases), and the logarithm of the ratio is thus log P. Log P values are well known and are determined according to a standard test that determines the concentration of monomer in a water/1-octanol separated mixture:

$$log {P}_{oct / wat} = log ( \frac{{[ solute ]}_{octanol}^{un \text{-} ionized}}{{[ solute ]}_{water}^{un \text{-} ionized}} )$$

wherein solute=tested monomer

There are also computer programs, commercially available, as well as on various internet sites, that will estimate the log P values for particular monomers.

Accordingly, monomers employed in preparing the hydrophilic polymeric chains which make the hydrophilic polymer network of the invention will typically have a log P value of less than about 1 or 0.5, and preferably will have a log P value of about 0.3, 0.1 or less (e.g., less than about −0.1, −0.3, −0.5 or less). For example, the following hydrophilic monomers have the following log P values: acrylic acid, about 0.35; 2-methoxyethylacrylate, about 0.45; and 2-hydroxyethyl-methacrylate, about 0.47. Other hydrophilic monomers and their log P values include, but are not limited to, acrylamide (about −0.67), 2-hydroxyethylacrylate (about −0.21), acrylic acid (0.35), methacrylic acid (0.93), N,N-dimethylacrylamide (−0.13), quaternized dimethylaminoethyl methacrylate, methacrylamide (−0.26), maleic acid (−0.48), maleic anhydride and its half esters, crotonic acid (0.72), itaconic acid (−0.34), acrylamide (−0.67), acrylate alcohols, hydroxyethyl methacrylate, diallyldimethyl ammonium chloride, vinyl ethers (such as methyl vinyl ether), maleimides, vinyl pyridine, vinylimidazole (0.96), other polar vinyl heterocycles, styrene sulfonate, allyl alcohol (0.17), vinyl alcohol (such as that produced by the hydrolysis of vinyl acetate after polymerization), salts of any acid or amine listed above, as well as mixtures thereof.

The hydrophobic monomer has a log P value in the range 1-3. Illustrative non-limitative examples are methyl methacrylate (1.38), n-butyl methacrylate (2.88), t-butyl methacrylate (2.5), styrene (2.95).

In one embodiment of the first aspect of the invention, the hydrophilic monomer(s) comprises functional groups selected from: acrylic, metacrylic and vinylic groups.

In one embodiment of the first aspect of the invention, the one or more hydrophilic monomer(s) are selected from, methacrylamide (MAAm), 2-methacryloyloxyethyl phosphorylcholine (MPC), SBMAAm, such as poly(ethyleneglycol) methacrylate, poly(ethylene glicol) dimethacrylate, and poly(ethylene glicol) diacrylate.

As mentioned in the first aspect of the invention, the hydrophilic polymeric chains, forming the random polymeric network, are directly bound to the surface of the substrate.

The term “directly bound” means that the network comprises polymeric chains which are bound to the substrate's surface with no inclusion of a specimen between the substrate and the polymeric chain.

It is well-known in the state of the art that the binding of a molecule to a surface of a particular material can be achieved when the surface is subjected to an activation treatment. There are well-known activation treatments such as plasma and corona treatment, chemical etching, priming with other chemicals, applying an adhesive film, or using atmospheric pressure plasma exposure. Subjecting the surface of the substrate to the activation treatment, a chemical modification of the surface is achieved. The nature of the chemical modification will depend on the particular activation treatment followed and the chemical nature of substrate.

The skilled person, making use of the general knowledge is able to select the more appropriate activation treatment depending on the substrate and the molecule to be bind to the substrate. Reagents and conditions to perform such activation treatment is part of the routine work of the skilled person in the art.

In another embodiment of the first aspect of the invention, the coated substrate as defined in the first aspect of the invention shows a reduction of the wear of at least 10% in the wear test described in example 15, or at least 10% in the wear described in example 17 including abrasive particles.

In one embodiment, all the chains forming the network are cross-linked. In another embodiment, about from 0.1 to 100% of the chains forming the network are cross-linked. In another embodiment, about from 1 to 20% of the chains forming the network are cross-linked. In another embodiment, about from 1 to 10% of the chains forming the network are cross-linked.

In a second aspect, the present invention provides a process for preparing the coated substrate as defined in the first aspect of the invention.

In a first step, the process of the second aspect comprises the activation of the surface of the substrate to be coated. As it has been mentioned above, there are well-known activation treatments such as plasma and corona treatment, chemical etching, priming with other chemicals, applying an adhesive film, physical abrasion, or using atmospheric pressure plasma exposure, among others.

Subjecting the surface of the substrate to the activation treatment, a chemical modification of the surface is achieved. The nature of the chemical modification will depend on the particular activation treatment and the chemical nature of substrate. The skilled person, making use of the general knowledge is able of selecting the more appropriate activation treatment depending on the substrate and the molecule to be covalently bound to the substrate. Reagents and conditions to perform such activation treatment is part of the routine work of the skilled person in the art.

In one embodiment, the activation treatment comprises a plasma activation surface treatment.

“Plasma activation” is a method of surface modification employing plasma processing, which improves surface adhesion properties of many materials including metals, glass, ceramics, a broad range of polymers and textiles and even natural materials such as wood and seeds. Many types of plasmas can be used for surface activation. However, due to economic reasons, atmospheric pressure plasmas found most applications. They include arc discharge, corona discharge, dielectric barrier discharge and its variation piezoelectric direct discharge, among others. In one embodiment, the activation treatment consists of subjecting the substrate's surface to low pressure plasma. The “low pressure plasma” protocol consists of the step of exciting a gas (N₂, O₂, air, Argon, hydrogen, water, . . . ) by energy (high frequency electric fields, usually between 2 electrodes) supplied in vacuum, generating energetic species (electrons, radicals, ions . . . ) that react with the substrate to be modified. There are three plasma effects: (a) micro-sandblasting, wherein the surface is removed by the ion bombardment; (b) chemical reaction, wherein there is the chemical reaction of the ionized gas with the surface; and (c) UV radiation, wherein UV radiation breaks down long-chain carbon compounds. The skilled person, using the general knowledge, is able to optimize, routinely, the parameters of pressure, power, time, and gas flow, among others, to achieve the surface activation.

In one embodiment, optionally in combination with any of the embodiments provided above or below, the activation step uses O₂ gas. In another embodiment, optionally in combination with any of the embodiments provided above or below, the activation step is performed applying to the surface O₂ gas flow at a pressure in the range from 0.001 to 1 mbar. In another embodiment, optionally in combination with any of the embodiments provided above or below, the activation step is performed applying to the surface O₂ gas flow at a pressure in the range from 0.1 to 0.8 mbar. In another embodiment, optionally in combination with any of the embodiments provided above or below, the activation step is performed applying to the surface O₂ gas flow 0.1-0.5 mbar. In another embodiment, optionally in combination with any of the embodiments provided above or below, the activation step is performed applying to the surface at O₂ gas flow from 20 to 400 mL/min. In another embodiment, optionally in combination with any of the embodiments provided above or below, the activation step is performed applying to the surface at O₂ gas flow from 200 to 320 mL/min. In another embodiment, optionally in combination with any of the embodiments provided above or below, the activation step is performed applying to the surface at O₂ gas flow at 30 or 240 mL/min. In another embodiment, optionally in combination with any of the embodiments provided above or below, the activation step is performed applying to the surface a 0.1-0.3 bar of O₂ gas flow at 30 mL/min. In another embodiment, optionally in combination with any of the embodiments provided above or below, the activation step is performed applying to the surface a 0.2 mbar of O₂ gas flow at 240 mL/min.

In another embodiment, optionally in combination with any of the embodiments provided above or below, the low pressure plasma treatment is performed at a frequency in the range from 5 kHz-30 MHz, at a power in the range from 10 to 400 W, and for a period of time in the range from 2 to 60 minutes. In another embodiment, optionally in combination with any of the embodiments provided above or below, the low pressure plasma treatment is performed at a frequency in the range from 30-50 kHz, at a power in the range from 150 to 300 W, and for a period of time in the range from 2 to 20 minutes. In another embodiment, optionally in combination with any of the embodiments provided above or below, the low pressure plasma treatment is performed at a frequency in the range from 35-45 kHz, at a power in the range from 200 to 280 W, and for a period of time in the range from 2 to 10 minutes. In another embodiment, optionally in combination with any of the embodiments provided above or below, the low pressure plasma treatment is performed at a frequency of 40 kHz, at a power of 270 W, for 5 minutes. Alternatively, in another embodiment, optionally in combination with any of the embodiments provided above or below, the low pressure plasma treatment is performed at a frequency in the range from 5-15 MHz, at a power in the range from 20 to 40 W, and for a period of time in the range from 10 to 55 minutes. In another embodiment, optionally in combination with any of the embodiments provided above or below, the low pressure plasma treatment is performed at a frequency in the range from 8-13 kHz, at a power in the range from 25 to 35 W, and for a period of time in the range from 15 to 50 minutes. In another embodiment, optionally in combination with any of the embodiments provided above or below, the low pressure plasma treatment is performed at a frequency of 12 MHz, at a power of 30 W, for 20 or 45 minutes.

In another embodiment, optionally in combination with any of the embodiments provided above or below, the activation step is performed applying 0.15 mbar of O₂ gas flow at 30 mL/min, at 12 MHz, 30 W and 20 min. In another embodiment, optionally in combination with any of the embodiments provided above or below, the activation step is performed applying 0.15 mbar of O₂ gas flow at 30 mL/min, at 12 MHz, 30 W and 45 min. In another embodiment, optionally in combination with any of the embodiments provided above or below, the activation step is performed applying 0.2 mbar of O₂ gas flow at 240 mL/min, at 40 KHz, 270 W and 5 min.

In a second step, the activated substrate is partially or totally coated with the hydrophilic random polymeric network. This step comprises (a) the in situ radical polymerization starting from a solution comprising the appropriate monomer(s) using a radical polymerization initiator; and (b) the cross-linking of the polymeric chains, both steps being performed in the presence of the activated substrate.

When the activated substrate is put in contact with the solution referred in step (b.1.), monomers of the solution anchor in the activated regions of the substrate resulting from step (a). Due to the radical polymerization initiator present in solution, the functional groups of the monomers are activated and react randomly with other monomers and those regions of the surface which have been activated (i.e., have been chemically modified), thus generating a random binding between the monomer and the surface of the substrate. In this way, the resulting polymers are directly bound to the surface of the substrate.

The solvent used in the solution referred in step (b.1.) is of polar nature. The polarity is given as the dielectric constant (the ratio of the electrical capacity of a capacitor filled with the solvent to the electrical capacity of the evacuated capacitor (at 20° C. unless otherwise indicated). Illustrative non-limitative examples are: isobutyl Alcohol (16.68), 2-Methoxy ethanol (16.93), n-butyl alcohol (17.51, at 25° C.), methyl ethyl ketone (18.51), isopropyl alcohol (19.92 (25° C.)), n-propyl alcohol (20.33 (25° C.)), acetone (20.7 (25° C.)), ethyl alcohol (24.55 (25° C.)), N-methylpyrrolidone (32.2 (25° C.)), methanol (32.70 (25° C.)), N,N-dimethylformamide (36.71 (25° C.)), acetonitrile (37.5), dimethyl acetamide (37.78 (25° C.)), dimethyl sulfoxide (46.68), propylene carbonate (64.9), and water (80.1, 20° C.).

In one embodiment, the solvent is water, ethanol, or a mixture thereof. In another embodiment, the solvent is water alone or a mixture of water and ethanol. In another embodiment, the solvent is water alone or a mixture of water and ethanol at 50% v/v.

In the present invention, the term “% volume/volume” means the volume of EtOH (expressed in mL) per 100 mL of solution.

The term “radical polymerization initiator” refers to compounds that can produce radical species under mild conditions and promote radical reactions. These compounds generally possess weak bonds-bonds that have small bond dissociation energies. Typical examples are halogen molecules (such as chlorine), azo compounds, and organic and inorganic peroxides (such as di-tert-butyl peroxide (tBuOOtBu), benzoyl peroxide (PhCOO), methyl ethyl ketone peroxide, and acetone peroxide is on rare occasions used as a radical initiator). Alternatively, the term “a radical polymerization initiator” refers to a physical agent with the ability of initiating polymerization, such as temperature or light (such as U.V.).

In one embodiment, optionally in combination with any of the embodiments provided above or below, the initiator is a peroxydisulfate salt. The peroxydisulfate ion, S₂O₈⁻² is a oxyanion. Illustrative non-limitative examples of salts include sodium persulfate (Na₂S₂O₈), potassium persulfate (K₂S₂O₈), and ammonium persulfate ((NH₄)₂S₂O₈). These salts are colorless, water-soluble solids that are strong oxidants. In one embodiment, optionally in combination with any of the embodiments provided above or below, the initiator is ammonium persulfate (APS). In another embodiment, optionally in combination with any of the embodiments provided above or below, the initiator is a combination of APS and heating of the solution. In another embodiment, optionally in combination with any of the embodiments provided above or below, the initiator is a combination of a peroxydisulfate salt and heating the solution to a temperature in the range from 50 to 100° C. In another embodiment, optionally in combination with any of the embodiments provided above or below, the initiator is a combination of APS and heating the solution to a temperature in the range from 50 to 100° C. In another embodiment, optionally in combination with any of the embodiments provided above or below, the radical polymerization initiator is in a percentage by weight from 0.1 to 10% with respect to the weight of monomers. In another embodiment, optionally in combination with any of the embodiments provided above or below, the radical polymerization initiator is in a percentage by weight from 0.5 to 1.0% with respect to the total weight of the monomers.

In one embodiment of the second aspect of the invention, optionally in combination with any of the embodiments provided above or below, the in situ polymerization comprises heating the solution to a temperature in the range from 50 to 100° C. for a period of time in the range from 1 to 3 hours. In one embodiment of the second aspect of the invention, optionally in combination with any of the embodiments provided above or below, the in situ polymerization comprises heating the solution to a temperature of 70° C. for a period of time in the range from 1 to 3 hours. In one embodiment of the second aspect of the invention, optionally in combination with any of the embodiments provided above or below, the in situ polymerization comprises heating the solution to a temperature in the range from 50 to 100° C. for 2 hours. In one embodiment of the second aspect of the invention, optionally in combination with any of the embodiments provided above or below, the in situ polymerization comprises heating the solution to a temperature of 70° C. for 2 hours.

In another embodiment of the second aspect of the invention, optionally in combination with any of the embodiments provided above or below, the concentration of monomer(s) in the solution referred in step (b.1.) is in the range from 0.5 to 50% weight.

The term “percentage (%) weight” refers to the percentage of the solute (such as monomer or radical initiator), expressed in grams, in relation to 100 g of solution. For example, when reference is made to 3% wt means that there are 3 grams of monomer per 100 g of the whole solution (solvent +monomer).

In another embodiment of the second aspect of the invention, optionally in combination with any of the embodiments provided above or below, the one or more monomer(s) are hydrophilic monomers having a logP value in the range from −1 to 1. In another embodiment of the second aspect of the invention, optionally in combination with any of the embodiments provided above or below, the one or more monomer(s) are selected from methacrylamide (MAAm), 2-methacryloyloxyethyl phosphorylcholine (MPC), SBMAAm, poly(ethylene) glycol methacrylate, poly(ethylene) glicol dimethacrylate, and poly(ethylene) glicol diacrylate.

In one embodiment of the second aspect of the invention, optionally in combination with any of the embodiments provided above or below, steps (b.1) and (b.2) are performed in a one-pot reaction and the solution referred in step (b.1.).

In the present invention, the term “molar ratio” refers to the number of moles of monomer(s) with respect to the number of moles of cross-linking agent.

In another embodiment of the second aspect of the invention, optionally in combination with any of the embodiments provided above or below, the cross-linking agent is selected from the group consisting of: methylmethacrylamide, BAm, PEGDA, PETA, GPTA, PRGDA, PEGDMA, DEGDMA, and BMAP.

In another embodiment of the second aspect of the invention, optionally in combination with any of the embodiments provided above or below, the radical polymerization initiator in the solution referred in step (b.1.) is in the range comprised from 0.01 to 20% weight with respect to the total weight of the monomers.

In another embodiment, optionally in combination with any of the embodiments provided above or below, step (b) is performed under mechanical pressure. In this way a more homogeneous distribution of the coating is achieved.

In another embodiment, optionally in combination with any of the embodiments provided above or below, the process further comprises a step of purification of the coated substrate and/or a drying step. In another embodiment, optionally in combination with any of the embodiments provided above or below, the purification step comprises soaking with a solvent. In one embodiment, the solvent is a polar solvent such as water, EtOH. The purification step can comprise, furthermore, the sonication of the coated substrate.

As mentioned above, due to the particular properties of the coated substrate of the first aspect of the invention, these substrates find use in several fields.

In this regards, medical devices coated with the formulation according to the present invention become lubricious when rewetted by contact with water or by introduction into a human or animal body, when brought into contact with body fluid. The hydrophilic coating for medical devices can optionally contain a drug for therapeutic purposes with or without elution. Alternatively, anti-microbials and bio-effecting agents can be chemically bonded into the hydrophilic coating for biostatic purposes. The hydrophilic coating according to the present invention can also have a chemically bonded radio-opaque substance to enhance X-Ray visibility of plastic or metallic medical devices during the process of introduction into the body or during an intended period of service time once it is implemented into the body. In addition to a quick initial lubricity, the hydrophilic lubricious coating of this invention is resistant to abrasion. Consequently, a catheter coated in accordance with the teachings of this invention will retain a lubricious surface for a long duration which is often required during the course of a surgical procedure.

Unlike catheters made of or coated with Teflon or silicones, catheters coated in accordance with the present invention are non-slippery when dry but become instantly slippery when wet. As a result, medical devices coated with the hydrophilic lubricious coatings of this invention are easier to handle and store.

In addition, due to the hydrophilicity of the coating, which is covalently bound to the surface of the substrate such as metal, glass or plastic surfaces, it is prevented water droplet formation on said surfaces when exposed to air of high humidity, to water vapor or when transferred from low temperature environment to higher temperature environment causing the surfaces usually to fog up. It also maintains good transparency on clear plastic or glass used as protective shields, windows, windshields, greenhouse panels, food packaging foils, goggles, optical glasses, contact lenses and the like. Due to the properties of the coating, the coated substrate of the invention can also found application in water filters (wherein hole's obstruction risk can be avoided or minimized).

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### List of Abbreviations

PEGMA-360 Poly(ethylene glycol) methacrylate (Mn 360)

PEGDMA-550 Poly(ethylene glycol) dimethacrylate (Mn 550)

UHMWPE Ultrahigh molecular weight polyethylene

ZTA Zirconia-toughened-alumina

XLPE Crosslinked polyethylene

PEEK-CFR Carbon-fiber-reinforced polyetheretherketone

MAAm Methacrylamide

MMA Methylmethacrylate

PEGDA-256 Poly(ethylene glycol) diacrylate (Mn 256)

DEGDMA Di(ethylene glycol) dimethacrylate

BMAP Bis[2-(methacryloyloxy)ethyl] phosphate

MPC 2-Methacryloyloxyethyl phosphorylcholine

SBMAAm [3-(Methacryloylamino)propyl]dimethyl(3-sulfopropyl)ammonium hydroxide] inner salt

BAm N,N′-Methylenebis(acrylamide)

PETA Pentaerythritol triacrylate

GPTA Glycerol propoxylate (1P0/0H) triacrylate

### Example 1: General Procedure for the Synthesis of a Coating Based on PEGMA-360 Covalently Cross-Linked with PEGDMA-550 on UHMWPE

UHMWPE with different roughness (Ra between 50 nm and 0.5 μm using confocal mycroscopy Leica DCM3D) and different morphologies (6 mm ball, cups 52/32, 18 cm² and 50 cm² flat surfaces, and 5 mm height 1 mm diameter cylinders) were first washed with water (and detergent if there are remnants of dirt), distilled (DI) water and ethanol.

After drying at room temperature overnight, the surface of the substrate was activated with low pressure plasma (0.2 mbar of O₂ at 240 mL/min) at a 40 KHz frequency and 270W during 5 min. An aqueous solution at 10% weight/volume, containing a mixture of PEGMA-360 as monomer, and PEGDMA-550 as cross-linker, at a molar ratio of 95:5 and 1% weight of APS with respect to the weight of the monomer and cross-linker, was added to the substrate (6 μL/cm²) and pressed with a mold to ensure the complete wetting and homogeneous distribution of the solution over the surface.

The polymerization was carried out by heating the substrates and the mold at 70° C. during 2 hrs. Then, the substrates were washed in excess water and sonicated (Bandelin, Sonorex Super) twice during 10 min. A further purification step consisted in placing the substrate in ethanol solution and sonicated during 10 min. Finally, the substrate was dried at room temperature overnight.

### Example 2 (comparative): Non Cross-Linked and Cross-Linked PEGMA-360 Based Coatings on UHMWPE

UHMWPE 18 cm² flat surfaces (Ra between 50 nm and 0.5 μm) were modified using the same protocol as in Example 1 but using the reaction mixture described in Table 1:

TABLE 1
Non cross-linked and cross-linked PEGMA-360
based coatings synthesized over UHMWPE flat surfaces.

| | Solid Content^{a} | PEGMA- | PEGDMA- | APS^{b} |
|---|---|---|---|---|
| Example | (%) | 360 (mmol) | 550 (mmol) | (%) |
| 2a | 10 | 90 | 10 | 1 |
| 2b | 10 | 80 | 20 | 1 |
| 2c | 10 | 60 | 40 | 1 |
| 2d | 30 | 80 | 20 | 1 |
| 2e | 30 | 95 | 5 | 1 |
| 2f | 10 | 100 | 0 | 1 |
| 2g | 20 | 100 | 0 | 1 |
| 2h | 30 | 100 | 0 | 1 |
| 2i | 40 | 100 | 0 | 1 |

^{a}% weight of monomer/volume solution
^{b}% weight of APS/weight of monomers

### Example 3: Coating Based on PEGMA-360 Crosslinked with PEGDMA-550 on ZTA

5 mm, 32 mm and 36 mm ZTA balls were modified using the same protocol as in example 1 but using an aqueous solution containing a mixture of 10 wt. % of PEGMA-360 as monomer, PEGDMA-550 as cross-linker, at a molar ratio of 95:5, and 1 wt. % of APS. The activation with plasma in this case lasted 10 min using the same conditions as in Example 1.

### Example 4: Coating Based on PEGMA-360 Crosslinked with PEGDMA-550 on Different Substrates Over Stainless Steel

6 mm balls and 3.5 cm² flat surfaces were modified using the same protocol as in example 3.

### Example 5: Coating Based on PEGMA-360 Crosslinked with PEGDMA-550 on Different Substrates Over Alumina

6 mm balls and 8.3 cm² flat surfaces were modified using the same protocol as in Example 3.

### Example 6: Coating Based on PEGMA-360 Crosslinked with PEGDMA-550 on XLPE

52/36 cups of XPE were modified using the same protocol as in Example 1.

### Example 7.—Coating Based on MAAm Physical Networks on PEEK-CFR

PEEK-CFR of different morphologies (28/40 cups and 3.8 cm² flat surfaces) were first washed with water (and detergent if needed), DI water and ethanol, as disclosed in Example 1.

After drying at room temperature overnight, the surface of the substrate was activated with low pressure plasma at 12 MHz frequency and 30 W power during (45 min) under (0.15 mbar) of O₂ (30 mL/min). An aqueous solution containing 20 wt. % of MAAm, as monomer, MMA as physical crosslinker, at a molar ratio 70:30, and 10 wt. % of APS, was added to the substrate (6 μL/cm²) and pressed with a mold to ensure the complete wetting and homogeneous distribution of the solution over the surface.

The polymerization was carried out by heating the substrates and the mold at 70° C. during 2 hrs. Then, the substrates were washed in excess water and sonicated twice during 10 min. A further purification step consisted in placing the substrate in ethanol solution and sonicated during 10 min. Finally, the substrate was dried at room temperature overnight.

### Example 8.—Coating Based on MAAm Physical Networks on Flat PEEK-CFR

PEEK-CFR (3.8 cm² flat surfaces) were first washed with water (and detergent if needed), DI water and ethanol, as disclosed in Example 1.

After drying at room temperature overnight, the surface of the substrate was activated with low pressure plasma at 12 MHz frequency and 30 W power during (45 min) under (0.15 mbar) of O₂ (30 mL/min). An aqueous solution containing between 15 wt. % of MAAm, as monomer, MMA as physical crosslinker, at a molar ratio 80:20, and 10 wt. % of APS, was added to the substrate (6 μL/cm²) and pressed with a mold to ensure the complete wetting and homogeneous distribution of the solution over the surface.

The polymerization was carried out by heating the substrates and the mold at 70° C. during 2 hrs. Then, the substrates were washed in excess water and sonicated twice during 10 min. A further purification step consisted in placing the substrate in ethanol solution and sonicated during 10 min. Finally, the substrate was dried at room temperature overnight.

### Example 9.—Coating Based on MAAm Physical Networks on ZTA

5 mm and 28 mm ZTA balls were modified using protocol of Example 7 but the activation with plasma in this case was at low pressure plasma (0.2 mbar of O₂ at 240 mL/min) at 40 KHz frequency and 270 W power during 10 min.

### Example 10.—Coating Based on Cross-Linked PEGMA-360 on UHMWPE Using Different Crosslinkers

UHMWPE 18 cm² flat surfaces were modified using the same protocol as in Example 1 but replacing the crosslinker of Example 1 by one selected from: PEGDA-256, DEGDMA and BMAP.

Example 11.—Coating Based on Cross-Linked MPC on UHMWPE Using Different Crosslinkers

UHMWPE 18 cm² flat surfaces (Ra between 50 nm and 0.5 μm) were modified using the same protocol as in Example 1 but using the reaction mixtures described in Table 2:

TABLE 2
Non cross-linked and cross-linked MPC based
coatings synthesized over UHMWPE flat surfaces.

| | Solid | | Cross- | | |
|---|---|---|---|---|---|
| | Content^{a} | MPC | linker | | APS^{b} |
| Example | (%) | (mmol) | (mmol) | Cross-linker | (%) |
| 11a | 5 | 100 | — | — | 1 |
| 11b | 10 | 100 | — | — | 1 |
| 11c | 20 | 100 | — | — | 1 |
| 11d | 40 | 100 | — | — | 1 |
| 11e | 10 | 95 | 5 | PEGDA-256 | 1 |
| 11f | 10 | 95 | 5 | DEGDMA | 1 |
| 11g | 10 | 95 | 5 | BMAP | 1 |
| 11h | 10 | 95 | 5 | PEGDMA-550 | 1 |
| 11i | 10 | 90 | 10 | PEGDMA-550 | 1 |
| 11j | 10 | 80 | 20 | PEGDMA-550 | 1 |
| 11k | 10 | 60 | 40 | PEGDMA-550 | 1 |

^{a}% weight of monomer/volume solution
^{b}% weight of APS/weight of monomers

### Example 12.—Coating Based on Cross-Linked MAAm on PEEK-CFR Using Different Crosslinkers

3.8 cm² PEEK-CFR flat surfaces were first washed with water (and detergent if needed), DI water and ethanol.

After drying at room temperature overnight, the surface of the substrate was activated with low pressure plasma at 12 MHz frequency and 30 W power during time in min (see Table 3) under (0.15 mbar) of O₂ (30 mL/min). An 50% ethanol aqueous solution containing a mixture of MAAm as monomer, a crosslinker selected from BAm, PEGDA-256, PETA and GPTA, at different molar ratios (see Table 3), and 10 wt. % of APS, was added to the substrate (6 μL/cm²) and pressed with a mold to ensure the complete wetting and homogeneous distribution of the solution over the surface.

The polymerization was carried out by heating the substrates and the mold at 70° C. during 2 hrs. Then, the substrates were washed in excess water and sonicated twice during 10 min. A further purification step consisted in placing the substrate in ethanol solution and sonicated during 10 min. Finally, the substrate was dried at room temperature overnight. The tested mixtures were:

TABLE 3
Different MAAm based coatings synthesized on PEEK-CFR.

| | Time in | Solid | | Cross- | | |
|---|---|---|---|---|---|---|
| | plasma | Content^{a} | MAAm | linker | Cross- | APSb |
| Example | (min) | (%) | (mmol) | (mmol) | linker | (%) |
| 12a | 30 | 5 | 97.5 | 2.5 | BAm | 10 |
| 12b | 45 | 20 | 95 | 5 | BAm | 10 |
| 12c | 45 | 5 | 97.5 | 2.5 | PEGDA-256 | 10 |
| 12d | 30 | 20 | 95 | 5 | PEGDA-256 | 10 |
| 12e | 45 | 20 | 97.5 | 2.5 | PETA | 10 |
| 12f | 30 | 5 | 95 | 5 | PETA | 10 |
| 12g | 30 | 20 | 97.5 | 2.5 | GPTA | 10 |
| 12h | 45 | 5 | 95 | 5 | GPTA | 10 |

^{a}% weight of monomer/volume solution
^{b}% weight of APS/weight of monomers

### Example 13.—Coating Based on SBMAAm on PEEK-CFR Using Different Crosslinkers

3.8 cm² PEEK-CFR flat surfaces were first washed with water (and detergent if needed), DI water and ethanol.

After drying at room temperature overnight, the surface of the substrate was activated with low pressure plasma at 12 MHz frequency and 30 W power during time in min (see Table 4) under (0.15 mbar) of O₂ (30 mL/min). An 50% ethanol aqueous solution containing a mixture of SBMAAm as monomer, a crosslinker selected from BAm, PEGDA-256, PETA and GPTA, at different molar ratios (see Table 4), and 10 wt. % of APS, was added to the substrate (6 μL/cm²) and pressed with a mold to ensure the complete wetting and homogeneous distribution of the solution over the surface.

The polymerization was carried out by heating the substrates and the mold at 70° C. during 2 hrs. Then, the substrates were washed in excess water and sonicated twice during 10 min. A further purification step consisted in placing the substrate in ethanol solution and sonicated during 10 min. Finally, the substrate was dried at room temperature overnight. The mixtures tested were:

TABLE 4
Different SBMAAm based coatings synthesized on PEEK-CFR.

| | Time in | Solid | | Cross- | | |
|---|---|---|---|---|---|---|
| | plasma | Content^{a} | SBMAAm | linker | Cross- | APS^{b} |
| Example | (min) | (%) | (mmol) | (mmol) | linker | (%) |
| 13a | 30 | 15 | 90 | 10 | BAm | 10 |
| 13b | 45 | 30 | 80 | 20 | BAm | 10 |
| 13c | 45 | 15 | 90 | 10 | PEGDA-256 | 10 |
| 13d | 30 | 30 | 80 | 20 | PEGDA-256 | 10 |
| 13e | 45 | 30 | 90 | 10 | PETA | 10 |
| 13f | 30 | 15 | 80 | 20 | PETA | 10 |
| 13g | 30 | 30 | 90 | 10 | GPTA | 10 |
| 13h | 45 | 15 | 80 | 20 | GPTA | 10 |

^{a}% weight of monomer/volume solution
^{b}% weight of APS/weight of monomers

### Example 14. Influence of Surface Roughness

Wear test: Wear tests were performed with a pin-on-disk CSM-THT tribometer at 1N loading and 1.1 Hz (6.9 rad/s) angular velocity at room temperature in simulated body fluid (SBF) with ˜20 mg/mL of BSA. The alumina pin had a spherical morphology with a diameter of 6 mm. Standard F732 (ASTM F732-00(2006), Standard Test Method for Wear Testing of Polymeric Materials Used in Total Joint Prostheses, ASTM International, West Conshohocken, Pa., 2006, www.astm.org) was taken into account as much as possible during the experiment. Wear for each substrate was calculated following G99 standard (ASTM G99-05, Standard Test Method for Wear Testing with a Pin-on-Disk Apparatus, ASTM International, West Conshohocken, Pa., 2005, www.astm.org) from three independent measurements after 100,000 cycles for UHMWPE Ra(<0.03 μm) and 10,000 cycles for UHMWPE Ra(>0.1 μm). Briefly, the wear track radius was measured in triplicates using the microscope, then the following equation was applied:

Volume loss (mm)³=π*wear track radius (mm)*(track width (mm))³/6*pin radius (mm)

The volume is transformed in weight loss by multiplying by the density of the material.

Results: The stability of the coating obtained following Example 1 using 10% wt. of the mixture of PEGMA-360 and PEGDMA-550 and a molar ratio of 95:5, was not affected by the roughness of the substrate.

### Example 15: Wear in Extreme Conditions—Cutting Edge

Wear test: Wear tests were performed with a pin-on-disk CSM-THT tribometer at 1N loading and 1.1 Hz (6.9 rad/s) angular velocity at room temperature in simulated body fluid (SBF) with ˜20 mg/mL of BSA. The UHMWPE pin had a cylindrical morphology with a diameter of 5 mm and 20 mm length. Standard F732 was taken into account as much as possible during the experiment. The experiments were carried out over 100,000 cycles. Wear for each substrate was calculated by gravimetry (difference in weight before and after the experiment a cleaning) in triplicates. The cleaning used was the same as described for the substrates before plasma polymerization. As control, 3 different uncoated pines were immersed in the SBF and cleaned using the same protocol (Entry 3). Coated cylinders following the procedure described in Example 1 of the mixture of PEGMA-360 and PEGDMA-550 and a molar ratio of 95:5 and coated alumina following the procedure described in Example 5 were tested.

The resulting wear for the bare UHMWPE cylinder against the coated alumina surface using the protocol described in Example 5 is 70% less (Entry 1) compared to the bare substrates (Entry 2).

TABLE 5

| Entry | Coating | Wear (μg) | Std. (μg) |
|---|---|---|---|
| 1 | Example 5 | 130 | ±100 |
| 2 | — | 400 | ±50 |
| 3 | Control | 40 | ±50 |

### Example 16.—Wear in Extreme Conditions—Third Body Abrasion Tests

Wear test: Wear tests were performed with a pin-on-disk CSM-THT tribometer at 1N loading and 1.1 Hz (6.9 rad/s) angular velocity at room temperature in simulated body fluid (SBF) with ˜20 mg/mL of BSA and with 1 mg/mL of 1 μm alumina particles (micropolish Buehler, Ref. 40-10079). The alumina pin had a spherical morphology with a diameter of 6 mm. Standard F732 was taken into account as much as possible during the experiment. Wear for each substrate was calculated following G99 standard from three independent measurements after 100,000 cycles.

UHMWPE was coated using the protocol described in Example 1 with 10% wt. of the mixture of PEGMA-360 and PEGDMA-550 and a molar ratio of 95:5 and alumina using the protocol described in Example 5. The substrates obtained were tested using the conditions explain above and compare with the bare substrates and the combination of a coated surface with a bare surface.

Results: When both surfaces were coated, the abrasive effect of the alumina particles was reduced by 55% compared to bare surfaces, equal to the reduction of wear observed when the coating was only applied over the UHMWPE substrate.

TABLE 6

| Coating over | Coating over | Wear | |
|---|---|---|---|
| Alumina | UHMWPE | (μg) | Std. (μg) |
| — | Example 1 | 18 | ±1 |
| Example 5 | Example 1 | 18 | ±1 |
| — | — | 42 | ±25 |

### Example 17.—Hip Simulator Study-UHMWPE

Wear test: Coated 52/32 UHMWPE cups following the protocol described in Example 1 and 32 mm ZTA balls using the protocol described in Example 3 were tested in the hip-simulator following standard conditions and using third body abrasion particles. The tests were conducted on a simulator fulfilling the requirements of ISO 14242-1 (ISO 14242-1:2014 Implants for Surgery—Wear of Total Hip-Joint Prostheses—Part 1: Loading and Displacement Parameters for Wear-Testing Machines and Corresponding Environmental Conditions for Test. ISO Copyright Office, Geneva, Switzerland).

It offers six wear stations and two reference stations to measure the soaking. Briefly, the samples subjected to the wear test were positioned with 30° inclination and the load was introduced through the embedding onto the cup and the head. Considering 10° inclination of the femoral component, the total inclination was 40°. The soaking samples were positioned upside-down (i.e. the cup beneath the head). Each test chamber was filled with a serum-based test liquid according to the ISO 14242-1:2014 standard (BSA30 g/L), which was kept at 37±1° C. using a thermostatic bath controlled with a temperature sensor. A fill level sensor controlled that the articulation partners remained wet at all time. The applied load and rotation simulate the conditions while walking. They were adjusted and controlled digitally. As control experiment, the same load profile was applied on the soaked samples, but without the motion. A load-motion cycle lasted one second and was repeated continually. After each 500′000 cycles, the test chambers were taken apart, cleaned, filled with fresh test liquid, reassembled and installed in another measuring station of the hip simulator. The weighing after washing using the protocol required by the ISO 14242-2 (ISO 14242-2:2000 Implants for Surgery—Wear of Total Hip-Joint Prostheses—Part 2: Methods of Measurements. ISO Copyright Office, Geneva, Switzerland) was done after each 500′000 cycles. This part of the test lasted 3 Million Cycles (MC). Afterwards, the test was continued with the addition of 0.15 g/L 1 μm alumina particles (micropolish Buehler, Ref. 40-10079) into the test liquid for another 2 MC. For the last 1 MC of totally 6 MC, 0.05 g of the same alumina particles was introduced directly between the articulating partners while reassembling the test chambers. With the addition of about 300 mL test liquid, this resulted in a similar particle concentration as before. In the case of the soaked samples, only 0.02 g of particles and about 120 mL test liquid were added.

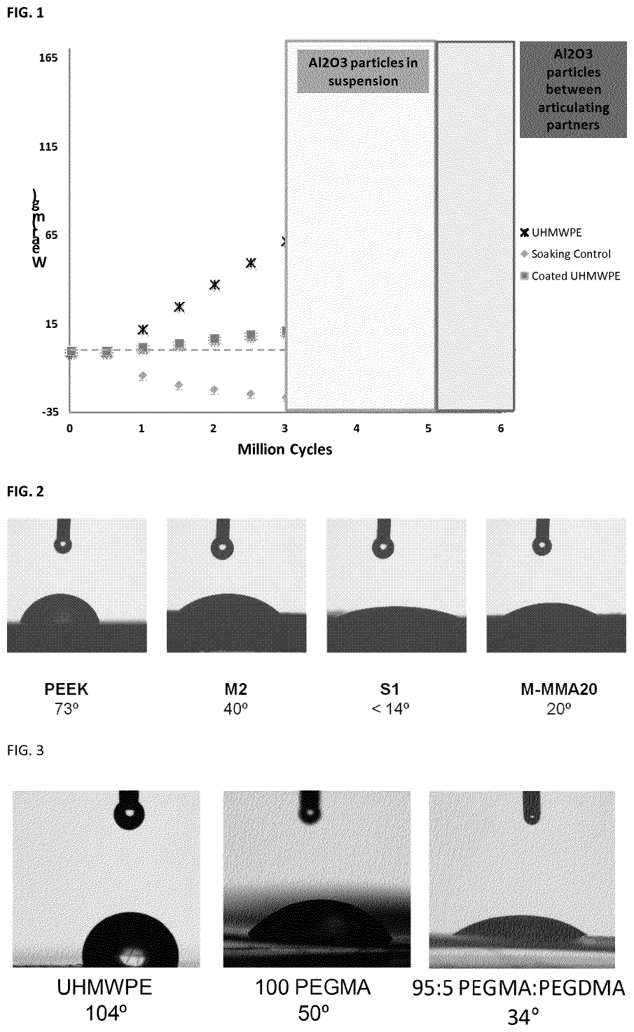

Results: The presence of the coating on UHMWPE and ZTA reduce by 80% the wear of the substrates compared to the uncoated ones. When abrasive particles were used the wear rate was 55% less (FIG. 1) when both substrates were coated.

### Example 18.—Hip Simulator Study-XLPE

Wear test: Coated 52/36 XLPE cups using the protocol described in Example 6 and 36 mm ZTA balls following the protocol described in Example 3 were tested in the hip-simulator following standard conditions and using third body abrasion particles. The tests were conducted on a simulator fulfilling the requirements of ISO 14242-1:2014 (as above).

It offers six wear stations and two reference stations to measure the soaking. The samples subjected to the wear test were positioned with 30° inclination and the load was introduced through the embedding onto the cup and the head. Considering 10° inclination of the femoral component, the total inclination was 40°. The soaking samples were positioned upside-down (i.e. the cup beneath the head). Each test chamber was filled with a serum-based test liquid according to the ISO 14242-1:2014 standard (BSA 30 g/L), which was kept at 37±1° C. using a thermostatic bath controlled with a temperature sensor. A fill level sensor controlled that the articulation partners remained wet at all time. The applied load and rotation simulate the conditions while walking. They are adjusted and controlled digitally. As control experiment, the same load profile is applied on the soaked samples, but without the motion. A load-motion cycle lasted one second and was repeated continually. After each 500′000 cycles, the test chambers were taken apart, cleaned, filled with fresh test liquid, reassembled and installed in another measuring station of the hip simulator. The weighing after washing using the protocol required by the ISO 14242-2 (see above) was done after each 500′000 cycles. This part of the test lasted 3 Million Cycles (MC). Afterwards, the test was continued with the addition of 0.15 g/L alumina 1 μm particles (micropolish Buehler, Ref. 40-10079) into the test liquid for another 2 MC. For the last 1 MC of totally 6 MC, 0.05 g of the same alumina particles was introduced directly between the articulating partners while reassembling the test chambers. With the addition of about 300 mL test liquid, this resulted in a similar particle concentration as before. In the case of the soaked samples, only 0.02 g of particles and about 120 mL test liquid were added.

Results: when the abrasive particles in suspension were added, the wear rate of the non-coated system increased as observed in the table. In contrast, the slope (wear rate) of the coated system was reduced when abrasive particles in suspension were used in the test indicating that the coating is able to protect the substrate in drastic conditions.

TABLE 7

| Coating over | Coating | Abrasive | Wear rate | Std. |
|---|---|---|---|---|
| XLPE | over ZTA | particles | (mg/MC) | (mg/MC) |
| — | — | — | 6 | ±3 |
| Example 6 | Example 3 | — | 6 | ±3 |
| — | — | CaSO₄ 1-2 μm | 9 | ±2 |
| Example 6 | Example 3 | Alumina 1 μm | 4 | ±2 |

Example 19.—Wear of coated PEEK-CFR substrates using the protocol described in Example 7 prepared at 20% mixture of MAAm:MMA and a molar ratio of 70:30 and ZTA coated following protocol described in Example 8.

Wear test: Wear tests were performed with a pin-on-disk CSM-THT tribometer at 1N loading and 1.1 Hz (6.9 rad/s) angular velocity at room temperature in simulated body fluid (SBF) with ˜20 mg/mL of BSA. The ZTA pin had a spherical morphology with a diameter of 6 mm. Standard F732 (as above) was taken into account as much as possible during the experiment. Wear for each substrate was calculated following G99 (as above) standard from three independent measurements after 100,000 cycles.

Results: The wear of the coated PEEK-CFR using the protocol described in Example 7 prepared at 20% comonomer mixture of MAAm:MMA and a molar ratio of 70:30 against bare ZTA was 37% lower compared to the bare surfaces. Furthermore, when the coated-ZTA following the protocol described in Example 9 against bare PEEK-CFR was used or both surfaces were coated, the wear obtained was 45% lower.

TABLE 8

| Coating over | Coating | | |
|---|---|---|---|
| PEEK | over ZTA | Wear (μg) | Std. (μg) |
| — | — | 12 | ±3 |
| Example 7 | — | 8 | ±1 |
| — | Example 9 | 7 | ±1 |
| Example 7 | Example 9 | 7 | ±1 |

As it can be derived, a remarkable improvement is achieved just coating one of the surfaces.

### Example 20.—Cytocompatibility of Example 1

TABLE 9
Coating compositions tested, prepared following
the protocol described in Example 1.

| Solid Content | PEGMA-360 | PEGDMA-550 | APS |
|---|---|---|---|
| (%) | (mmol) | (mmol) | (%) |
| 10 | 95 | 5 | 1 |
| 10 | 80 | 20 | 1 |
| 30 | 95 | 5 | 1 |
| 30 | 80 | 20 | 1 |

Biocompatibility test: Cytotoxicity of the coatings was tested in the presence of HeLa cells (ECACC, Cat. No. 93021013) using the elution method based on the requirements of the Biological evaluation of medical devices—Part 5: Tests for in vitro cytotoxicity (ISO 10993-30 5:2009). A cell extract was prepared from a cell culture media (extraction conditions: 3cm²/mL (volume of solution employed per area), 24 h, 37° C.). HeLa Cells were cultured at a density of 10,000 cell/cm² for 24 hours to allow cell attachment. Cell cultures were maintained at 37° C. in an incubator with 95% humidity and 5% of CO₂. After 24 hours cell culture media was replaced with extraction media and cultured for another 24, 48 and 72 hours. Triton X-100 and non-treated HeLa cells were used as positive and negative controls. MTS assay (colorimetric) quantified viable cells.

Results: The coatings don't generate toxic leachables and are cytocompatible.

### Example 21.—Cytocompatibility of Some of the Coatings Prepared on PEEK Surfaces

### Coating Preparation

TABLE 10
Coating compositions tested, prepared following the Examples listed.

| | | | | | Monomer: | |
|---|---|---|---|---|---|---|
| | | | | | Cross- | |
| | | Solid | | | Linker | |
| | | Content | | | (mmol: | APS |
| | Code | (%) | Monomer | Crosslinker | mmol) | (%) |
| Example | S1 | 15 | SBMAm | BAm | 95:5 | 10 |
| 13 | | | | | | |
| Example | M2 | 15 | MAAm | PEGDA-256 | 95:5 | 10 |
| 12 | | | | | | |
| Example | M2015 | 20 | MAAm | MMA | 85:15 | 10 |
| 8 | | | | | | |

Biocompatibility test: Cytotoxicity of the material was tested in the presence of HeLa Cells based on the requirements of the ISO 10993 Part 5 (Elution method). A cell extract was prepared from a cell culture media (extraction conditions: 3cm²/mL (volume of solution employed per area), 24 h, 37° C.). HeLa cells were cultured at a density of 10,000 cell/cm^{2 for} 24 hours to allow cell attachment. Cell cultures were maintained at 37° C. in an incubator with 95% humidity and 5% of CO₂. After 24 hours cell culture media was replaced with extraction media and cultured for another 24 hours. Triton X-100 and non- treated Hela cells were used as positive and negative controls. MTS assay (colorimetric) quantified viable cells.

Results: Under conditions of this study, the test extracts showed no evidence of causing cell lysis or cytotoxicity.

### Example 22.—Hydrophilicity of Some of the Coatings Prepared on PEEK

### Coating Preparation

TABLE 11
Coating compositions tested following the Examples listed.

| | | | | | Monomer: | |
|---|---|---|---|---|---|---|
| | | | | | Cross- | |
| | | Solid | | | Linker | |
| | | Content | | | (mmol: | APS |
| | Code | (%) | Monomer | Crosslinker | mmol) | (%) |
| Example | S1 | 15 | SBMAm | BAm | 95:5 | 10 |
| 13 | | | | | | |
| Example | M2 | 15 | MAAm | PEGDA-256 | 95:5 | 10 |
| 12 | | | | | | |
| Example | M2015 | 20 | MAAm | MMA | 85:15 | 10 |
| 8 | | | | | | |

Contact angle measurement: The static contact angle was acquired on a CAM 200 at room temperature, equipped with a drop shape analysis system and a camera. A droplet of 5 μL of deionized water was used as probe liquid. Three different positions of the same coated surface were measured for each sample to get an average.

Results: Contact angle obtained in the coated samples are smaller than in the untreated surface (FIG. 2)

### Example 23.—Hydrophilicity of Coatings Prepared on UHMWPE

### Coating Preparation

TABLE 12
Tested coating compositions were prepared
following the Examples 1 and 2:

| | | Solid | | | |
|---|---|---|---|---|---|
| | | Content | PEGMA- | PEGDMA- | APS |
| | Code | (%) | 360 (mmol) | 550 (mmol) | (%) |
| Example 2f | PEG | 10 | 100 | 0 | 1 |
| Example 1 | 95:5 | 10 | 95 | 5 | 1 |

Contact angle measurement: The static contact angle was acquired on a CAM 200 at room temperature, equipped with a drop shape analysis system and a camera. A droplet of 5 μL of deionized water was used as probe liquid. Three different positions were measured for each sample to obtain an average value.

Results: Contact angle obtained in the coated samples following the protocol described in Table 12 are lower compared to the untreated surface. Furthermore, the coated surface following the protocol described in Example 1 with the conditions described in the Table 12, that corresponds with a hydrogel coating (crosslinked structure) is more hydrophilic than the linear polymer coating (Example 2). See FIG. 3.

### Example 24.—Antifouling Performance of Coating Prepared on UHMWPE

### Coating Preparation

TABLE 13
Coating compositions tested,
prepared following the Examples listed.

| | | Solid | | | |
|---|---|---|---|---|---|
| | | Content | PEGMA- | PEGDMA- | APS |
| | Code | (%) | 360 (mmol) | 550 (mmol) | (%) |
| Example 2f | PEG | 10 | 100 | 0 | 1 |
| Example 1 | 95:5 | 10 | 95 | 5 | 1 |

Antifouling test: A protocol was set up based on the bicinchoninic acid (BCA) method (Basic Protein and Peptide Protocols, Volume 32 of the series Methods in Molecular Biology™ pp 5-8). The P-6 plates containing the coated UHMWPE substrates, the bare substrate and the control without surface, were filled with PBS. All the substrates and the control were equilibrated in PBS during 24 h at room temperature. The plates were then filled with bovine serum albumin (BSA) solution (4.5 g/L) in PBS for 24 hours at 20° C. Then, the plates were rinsed two times with distilled water to remove the non-adsorbed proteins. The plates were filled with a 2% sodium dodecyl sulphate (SDS) aqueous solution and shaken at room temperature for 2 hours to remove the absorbed BSA on the surface. Triplicates of these solutions were placed in a new P-24 well and analyzed using a protein analysis kit (microBCA protein assay kit, 23235; Thermo Fisher Scientific Inc.) based on BCA method to determine the BSA concentration in SDS solution for the quantification of BSA adsorbed on the film. The experiment was performed in triplicate for each substrate.

Results: 40% less BSA absorption is observed in coated UHMWPE, when the coating is the Example 1 with the conditions described in the Table 5 that corresponds with a hydrogel coating (crosslinked structure). The BSA absorption is lower than with the UHMWPE coated with the protocol described in Example 2f.

TABLE 14

| Coating over | Amount of proteins | |
|---|---|---|
| UHMWPE | attached (μg/cm²) | Std. (μg/cm²) |
| — | 12 | ±3 |
| Example 2f | 9 | ±2 |
| Example 1 | 7 | ±1 |

### Example 25.—Thickness of a Coating Prepared Following the Protocol Described in Example 4

SEM: FE-SEM images (Zeis, Ultra Plus) of the samples were taken at an accelerating voltage of 20 kV over dried samples after gold sputtering using a Bal-Tec SCD 005 sputter

Results: Coating thickness is about 300 nm in dry state.

### Example 26.—Thickness of a Coating Prepared Following the Protocol Described in Example 1

SEM: A cross-section of the coated UHMWPE was observed under eSEM. The images of the samples were taken at an accelerating voltage of 20 kV over the sliced samples in dry and wet conditions (after 7 days in demi water). The coating swelling capability was observed by atmospheric pressure SEM (Environmental Scanning-electron Microscope (eSEM-FEI Quanta 250)) where images acquired at 100% of humidity, 750 Pa pressure and 1° C. of temperature. Then, humidity in the chamber was reduced to 45% of humidity (311 Pa pressure and 1° C. of temperature) to see the decrease of the thickness of the coating by drying the sample.

Results: Coating thickness is about 7 μm to 8 μm in wet state (100% humidity), 0.8-1 μm (45% humidity). In dry state is not measurable.

### Citation List

D. Xiong et al, “Influence of surface PMPC brushes on tribological and biocompatibility properties of UHMWPE”, Applied Surface Science, 2014, v. 298, 56-61,

PAC, 1996, 68, 2287 (Glossary of basic terms in polymer science (IUPAC Recommendations 1996)) on page 2289;

Standard F732 (ASTM F732-00(2006), Standard Test Method for Wear Testing of Polymeric Materials Used in Total Joint Prostheses, ASTM International, West Conshohocken, Pa., 2006, www.astm.org);

G99 standard (ASTM G99-05, Standard Test Method for Wear Testing with a Pin-on-Disk Apparatus, ASTM International, West Conshohocken, Pa., 2005, www.astm.org);

ISO 14242-1 (ISO 14242-1:2014 Implants for Surgery—Wear of Total Hip-Joint Prostheses—Part 1: Loading and Displacement Parameters for Wear-Testing Machines and Corresponding Environmental Conditions for Test. ISO Copyright Office, Geneva, Switzerland;

ISO 14242-2 (ISO 14242-2:2000 Implants for Surgery—Wear of Total Hip-Joint Prostheses—Part 2: Methods of Measurements. ISO Copyright Office, Geneva, Switzerland); and

Biological evaluation of medical devices—Part 5: Tests for in vitro cytotoxicity (ISO 10993-5:2009.

1. A partially or totally coated substrate, the coating comprising a water-absorbing hydrophilic random polymeric network comprising hydrophilic cross-linked polymeric chains which are directly bound to the surface of the substrate, said polymeric chains comprising one or more hydrophilic monomer(s).

2. The partially or totally coated substrate according to claim 1, wherein the hydrophilic monomer(s) comprises functional groups selected from: acrylic, methacrylic and vinylic groups.

3. The partially or totally coated substrate according to claim 1, wherein the polymeric chains comprise one or more of the following hydrophilic monomers: methacrylamide (MAAm), 2-methacryloyloxyethyl phosphorylcholine (MPC), SBMAAm, poly(ethylene) glycol methacrylate, poly(ethylene) glycol dimethacrylate, and poly(ethylene) glycol diacrylate.

4. The partially or totally coated substrate according to claim 1, wherein the substrate is of a material selected from ultrahigh molecular weight polyethylene (UHMWPE), vitamin E doped UHMWPE, Zirconia-toughened-alumina (ZTA), Crosslinked polyethylene (XLPE), and carbon-fiber-reinforced polyetheretherketone (PEEK-CFR).

5. The partially or totally coated substrate according to claim 1, the substrate being a medical device.

6. A process for preparing a partially or totally coated substrate according to claim 1, which comprises the steps of:
(a) subjecting substrate's surface to an activation surface treatment,
(b) partially or totally coating the activated surface resulting from step (a) by:
b.1. performing an in situ radical polymerization starting from a solution comprising the appropriate monomer(s), using a radical polymerization initiator, and
b.2. cross-linking the polymeric chains,
being carried out steps b.1 and b.2. in the presence of the activated surface resulting from step (a).

7. The process according to claim 6, wherein step (a) comprises subjecting substrate's surface to a plasma activation surface treatment.

8. The process according to claim 6, wherein step (a) is performed applying to the surface a O₂ gas flow in the range from 20 to 400 mg/mL at a pressure in the range from 0.001 to 1 mbar, and a discharge with a frequency of 5kHz-30 MHz, with a potency in the range from 10 to 400 W, and for a period of time in the range from 2 to 60 minutes.

9. The process according to claim 6, wherein steps (b.1) and (b.2) are performed in a one-pot reaction and the solution referred in step (b.1.) comprises, in addition to the appropriate monomer(s) and radical polymerization initiator, a cross-linking agent.

10. The process according to claim 6, wherein the in situ polymerization comprises heating the solution to a temperature in the range from 50 to 100° C. for a period of time in the range from 1 to 3 hours.

11. The process according to claim 6, wherein the solution referred in step b.1. further comprises a peroxydisulfate salt.

12. The process according to claim 6, wherein the concentration of monomer(s) in the solution referred in step (b.1.) is in the range from 0.5 to 50% weight/volume.

13. The process according to claim 6, wherein the concentration of radical polymerization initiator in the solution referred in step (b.1.) is in the range comprised from 0.01 to 20% weight/volume.

14. (canceled)

15. The partially or totally coated substrate according to claim 1, wherein the polymeric chains comprise one or more of the following hydrophilic monomers:
methacrylamide (MAAm), 2-methacryloyloxyethyl phosphorylcholine (MPC), SBMAAm, poly(ethylene) glycol methacrylate, poly(ethylene) glycol dimethacrylate, and poly(ethylene) glycol diacrylate; and the substrate is of a material selected from ultrahigh molecular weight polyethylene (UHMWPE), vitamin E doped UHMWPE, Zirconia-toughened-alumina (ZTA), Crosslinked polyethylene (XLPE), and carbon-fiber-reinforced polyetheretherketone (PEEK-CFR).

16. The partially or totally coated substrate according to claim 1, wherein the substrate is an implant such as a prosthesis, particularly an articulate prosthesis.

17. The process according to claim 6, wherein:
step (a) comprises subjecting substrate's surface to a plasma activation surface treatment,
the solution referred in step (b.1.) comprises, in addition to the appropriate monomer(s) and radical polymerization initiator, a cross-linking agent; and
steps (b.1) and (b.2) are performed in a one-pot reaction.

18. The process according to claim 17, wherein the in situ polymerization comprises heating the solution to a temperature in the range from 50 to 100° C. for a period of time in the range from 1 to 3 hours.

19. The process according to claim 17, wherein the concentration of monomer(s) in the solution referred in step (b.1.) is in the range from 0.5 to 50% weight/volume.

20. The process according to claim 17, wherein the concentration of radical polymerization initiator in the solution referred in step (b.1.) is in the range comprised from 0.01 to 20% weight/volume.
